(19) United States
(12) Patent Application Publication
(10) Pub. No.: US 2008/0147082 A1
(43) Pub. Date: Jun. 19, 2008

(54) INJECTOR APPARATUS FOR USE WITH INTRAOCULAR LENSES AND METHODS OF USE

(76) Inventor: Joel Pynson, Toulouse (FR)

Correspondence Address: Bausch & Lomb Incorporated, One Bausch & Lomb Place, Rochester, NY, 14604-2701, US

(21) Appl. No.: 11/610,057

(22) Filed: Dec. 13, 2006

(51) Int. Cl. A61F 9/013; A61B 19/02

(52) U.S. Cl. 606/107; 206/438

(57) ABSTRACT

An IOL injector comprising a plunger and an injector body having an axis, the IOL injector comprising a distal body portion having a first lumen disposed therethrough, and a distal cutting surface disposed proximate a proximal end of the distal portion and extending proximally from said first lumen into a space into which a container is to be received. In some embodiments, a proximal body portion is included with a second lumen disposed therethrough. In such embodiments, the proximal portion and the distal portion are separated along the axis by a gap.

### FIELD OF INVENTION

The present invention relates to intraocular lens injector loading apparatus and methods of use, and more particularly to intraocular lens injector apparatus and methods for loading an intraocular lens (IOL) disposed in a container.

### BACKGROUND OF THE INVENTION

IOLs are artificial lenses used to replace natural crystalline lenses of patients' when their natural lenses are diseased or otherwise impaired. Under some circumstances a natural lens may remain in a patient's eye together with an implanted IOL. IOLs may be placed in either the posterior chamber or the anterior chamber of an eye.

IOLs come in a variety of configurations and materials. Various instruments and methods for implanting such IOLs in an eye are known. Typically, an incision is made in a patient's cornea and an IOL is inserted into the eye through the incision. In one technique, a surgeon uses surgical forceps to grasp the IOL and insert it through the incision into the eye. While this technique is still practiced today, more and more surgeons are using IOL injectors, which offer advantages such as affording a surgeon more control when inserting an IOL into an eye and permitting insertion of IOLs through smaller incisions. Relatively small incision sizes (e.g., less than about 3 mm) are preferred over relatively large incisions (e.g., about 3.2 to 5+mm) since smaller incisions have been attributed with reduced post-surgical healing time and reduced complications such as induced astigmatism.

In order for an IOL to fit through a small incision, it is typically folded and/or compressed prior to entering the eye where it will assume its original unfolded/uncompressed shape. Since IOLs are very small and delicate articles of manufacture, great care is taken in their handling, both as they are loaded into an injector and as the lenses are injected into patients' eyes.

It is desirable that an IOL be expelled from the tip of the IOL injector and into the eye in an undamaged condition and in a predictable orientation. Should an IOL be damaged or expelled from the injector in an incorrect orientation, a surgeon may need to remove or further manipulate the IOL in the eye, possibly resulting in trauma to the surrounding tissues of the eye. To achieve proper delivery of an IOL, consistent loading of the IOL into the injector device with a limited opportunity for damaging the IOL is desirable.

Hydrophilic IOLs present particular challenges due to the need to maintain the lenses in a sterile solution prior to insertion into a patient's eye. Various IOL injectors and other devices have been proposed which attempt to address issues related to loading hydrophilic and/or hydrophobic IOLs into injectors, yet there remains a need for an IOL injector that improves consistency of loading and reduces the likelihood of damage to an IOL.

### SUMMARY

An aspect of the invention is directed to an IOL injector comprising an injector body, comprising (i) a first body portion having a first lumen disposed therethrough, a proximal end, a distal end configured to deposit an IOL into an eye, and (ii) a first cutting surface coupled to the first body portion, the first cutting surface being disposed proximate the proximal end of the first body portion and extending proximally of said first lumen. The IOL injector also typically comprises a plunger coupled to the injector body, the plunger having a longitudinal axis.

In some embodiments, the first body portion is suitably shaped to fold the IOL as the IOL passes therethrough. The injector may further comprise a guide coupled to the first body portion configured to facilitate coupling of a container to the injector. The injector body may include an attachment device such that a container can be connected to the injector body. In some embodiments, the first cutting surface is attached to the first body portion. The first cutting surface may be integrally formed with the first body portion.

In some embodiments, the proximal end of the first body portion is angled so as to be other than perpendicular to the axis. The first cutting surface may be disposed inside of a profile of the first lumen. The injector may further comprise a second body portion having a second lumen disposed therethrough, the second body portion and the first body portion being separated along the axis by a gap, the second body portion being disposed in a location that is more proximate than the first body portion, and the first and second lumens aligned such that the plunger can extend through the first and second lumens.

An aspect of the invention is directed to an IOL injector comprising A) a plunger having a longitudinal axis; and B) an injector body, comprising (i) a proximal body portion having a first lumen disposed therethrough and a distal body portion having a second lumen disposed therethrough, the proximal body portion and the distal portion being separated along the axis by a gap, and the first and second lumens aligned such that the plunger can extend through the first and second lumens, and (ii) a distal cutting surface disposed proximate an end of the distal portion and extending into said gap.

The IOL injector may further comprise a proximal cutting surface disposed proximate an end of the proximal body portion and extending into the gap. The IOL injector may further comprise a connector configured and arranged to couple the proximal body portion and the distal body portion together. The injector may further comprise a guide configured to facilitate movement of a container into a position between the proximal body portion and the distal body portion.

In some embodiments, the guide is attached to a connector, the connector being configured and arranged to couple the proximal body portion and the distal body portion together. The guide may be integrated with the connector. The guide may be disposed on at least one of the proximal and distal body portions. In some embodiments, at least one of the cutting surfaces is disposed inside a profile of a respective one of the first and second lumens. The proximal body portion may be rotationally complete. In some embodiments, at least one of the proximal end of the distal body portion and the distal end of the proximal body portion is angled so as to be other than perpendicular to the axis.

Another aspect of the invention is directed to a container for use with an IOL injector body, comprising: a wall defining a container lumen, a first open end, and a second open end; a first end cover and a second end cover configured and arranged to seal said first open end and said second open end, respectively; and an IOL disposed inside said container.

The IOL may be disposed in a holder, the holder being disposed in the container. The first open end and the second open end may be angled relative to one another. The container may further comprise a plunger holding apparatus.

Yet another aspect of the invention is directed to a method of loading an IOL injector with an IOL, the method comprising A.) providing a sealed container containing an IOL, the container comprising a first end cover to seal a first end of the container and a second end cover to seal a second end of the container, and a container lumen; B) moving the container into a position proximate an injector body defining a first injector body lumen such that the container lumen and the first injector body lumen at least partially overlap such that a plunger could extend through the first injector body lumen and the container lumen, and C) during the step of moving the container into a position proximate the injector body, cutting at least one of the first end cover and the second end cover with a first cutting surface coupled to the injector body.

In some embodiments, the step of moving the container further comprises sliding the container along a guide. The method may further comprise breaching the other of the first end cover and the second end cover with a plunger tip. The method may further comprise snapping the container into a final position relative to the injector body. In some embodiments, the method further comprises moving the container into a position proximate a second injector body lumen such that the container lumen and the second injector body lumen at least partially overlap such that a plunger could extend through the first and second injector body lumens and the container lumen. In some embodiments, the method further comprises a step of, during the step of moving the container into a position proximate the injector body, cutting the other of the first end cover and the second end cover with a second cutting surface coupled to the injector body

As used herein, the term “distal” refers to a direction toward the portion of an injector through which a lens enters a patient's eye from the injector. As used herein, the term “proximal” refers to a direction opposite of the distal direction.

### BRIEF DESCRIPTION OF THE DRAWINGS

Illustrative, non-limiting embodiments of the present invention will be described by way of example with reference to the accompanying drawings, in which same reference numbers are used to designate same or similar components in different figures, and in which:

FIG. 1 is a perspective view of an example of an embodiment of an IOL injector in which an injector body and a container are illustrated as separated from one another;

FIG. 2 is a perspective view of the embodiment of the IOL injector of FIG. 1 in which the injector body and the container are interacting to approach a complete IOL injector lumen;

FIG. 3A is a perspective view of the embodiment of the IOL injector of FIG. 1 in which the injector body and the container are interacting to approach a complete IOL injector lumen, and in which cutting surfaces of the injector body are cutting end covers of the container;

FIG. 3B is an expanded view of the IOL injector body and container of FIG. 3A in which interaction between the proximal cutting surface of the injector body and the proximal end cover of the container is beginning;

FIG. 3C is an expanded view of the IOL injector body and container of FIG. 3A in which interaction between the proximal cutting surface of the injector body and the proximal end cover of the container is substantially complete;

FIG. 4 is a perspective view of the IOL injector of FIG. 1 in which a complete injector lumen is formed by the injector body and the container;

FIG. 5 is a perspective view of the IOL injector of FIG. 1 in which the IOL is ejected from an injector having a completed injector lumen;

FIG. 6 is a top view of another example of an embodiment of an IOL injector according to aspects of the invention;

FIG. 7 is a top view of another example of an embodiment of an IOL injector according to aspects of the present invention in which an injector body and a container are separated from one another;

FIG. 8 is a perspective view of another embodiment of an IOL container;

FIG. 9 is a perspective view of the IOL container of FIG. 8 and a proximal body portion of an IOL injector;

FIG. 10 is a perspective view of the IOL container of FIG. 8, a proximal body portion and a distal body portion an IOL injector; and

FIG. 11 is a perspective view of the IOL container of FIG. 8, a proximal body portion and a distal body portion assembled to form a completed injector lumen.

### DETAILED DESCRIPTION

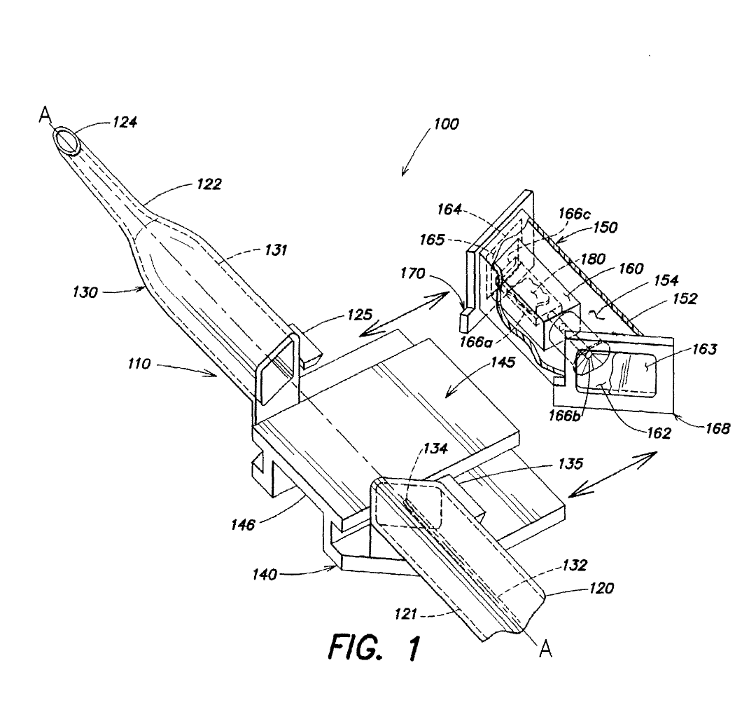

FIG. 1 is a perspective view of an example of an embodiment an IOL injector 100 according to aspects of the present invention. The IOL injector comprises an injector body 110 and a container 150 (e.g., a vial) that are capable of being coupled together. Injector body 110 comprises a proximal body portion 120 having a first lumen 121 disposed therethrough, and a distal body portion 130 having a second lumen 131 disposed therethrough. The distal body portion includes a cartridge 122 suitably shaped to fold and/or compress an IOL as the IOL passes through the cartridge. The IOL passes through the cartridge to an end 124 where the IOL is delivered into an eye. The end is sized to enter a relatively small incision in a patient's eye. Although the illustrated embodiment of the IOL injector includes a cartridge to fold and/or compress the IOL, an injector according to aspects of the present invention may be provided with no such apparatus. For example, a compressor drawer may be included to fold and/or compress the IOL.

IOL injector 100 comprises a plunger 132 extending along a longitudinal axis A-A. Axis A-A extends through the first lumen and the second lumen. Proximal portion 120 and the distal portion 130 are separated along the axis by a gap 145. A connector 140 is configured and arranged to couple the proximal body portion and the distal body portion together. The connector may have any suitable construction that couples the proximal body portion and the distal body portion in a manner that allows for a container (such as container 150) to be introduced between the portions.

In some embodiments, a guide 146 (e.g., a rail having one or more portions raised relative to the connector) is provided to facilitate coupling of the container to the injector body and movement of the container into a substantially aligned position between portions 120, 130. The guide may be attached to the connector, and may be integrated with connector to form a single part. However, injector embodiments that include a guide are not so limited, and such a guide may be formed as a separate component or omitted. In some embodiments, a guide may be located on one or both of the proximal and distal body portions rather than on the connector. Any suitable coupling mechanism may be provided on the injector body to receive the container, thereby maintaining the container during insertion of an IOL into a patient's eye. It is to be appreciated that the coupling may be by direct connection (e.g., using a mechanical or magnetic attachment mechanism) to one or both of the injector body portions. Alternatively, the coupling can be indirect through the guide or connector. Although gap 145 is illustrated as being bounded on the bottom (i.e., the gap is bounded by guide 146), a gap can be provided in any suitable configuration that permits introduction of a container between the proximal body portion and the distal body portion.

The first and second lumens are aligned such that the plunger can extend through the first and second lumens. In some embodiments, the lumens are configured and arranged such that axis A-A extends substantially through the centers of the proximate portion and the second portion. However, such an alignment is not necessary, and the centers may be offset from one another.

During actuation of the plunger, a tip 134 of the plunger pushes an IOL through the distal portion of the lumen and into an eye through end 124. The tip may have any suitable shape, such as a forked shape or may be soft tip (e.g., a silicone tip) having a flat, concave or convex shape.

It is to be appreciated that proximal body portion 120 may have any suitable configuration such that a plunger is maintained prior to actuation, and such that the plunger can be actuated to push an IOL into an eye. In some embodiments, lumen 121 may be smaller in cross section than lumen 131. It is to be further appreciated that the smaller size of the proximal body portion is possible because the primary function of the proximal body portion is to maintain the plunger 132 prior to actuation and allowing the plunger to be actuated. Proximal body portion 120 can be rotationally complete such that lumen 121 is a closed structure. Alternatively, proximal body portion 120 can have one or more open portions where the plunger is not surrounded by proximal body portion.

FIG. 1 also illustrates an example of an embodiment of container 150 for use with an IOL injector body having a gap, such as injector body 110 shown in FIG. 1. Container 150 comprises a wall 152 defining a container lumen 154, a proximal open end 162, and a distal open end 164. A proximal end cover 163 and a distal end cover 165 are configured and arranged to seal said first open end and said second open end, respectively. A schematically illustrated IOL 180 is disposed inside container 150.

In some embodiments, a hydrophilic IOL 180 is disposed inside container 150. It is to be appreciated that, by sealing container 150, IOL 180 can be maintained sterile and/or hydrated. In embodiments in which the IOL is to remain hydrated, a fluid is sealed inside the container along with the IOL. Typically, the fluid is provided in sufficient quantity such that the IOL is immersed in the fluid regardless of the orientation of the container.

IOL 180 may be maintained in a location within the container by any suitable apparatus such that the plunger tip can urge the IOL into an eye upon actuation. For example, IOL 180 may be disposed in a holder 160 as illustrated in FIG. 1. (Wall 152 is illustrated as partially cut away so that holder 160 is visible.) The holder may provide a channel 166a for receiving plunger 132. The holder may also suitably constrain the lens. In such embodiments, as the plunger extends through the channel, the plunger interacts with the lens in a predictable manner. In some embodiments, at the proximal end of the holder has a funnel shape to guide the plunger tip into the channel. End cover 163 is illustrated as partially cut away such that hole 166b is visible.

A hole 166c is located at the distal end of the channel. Hole 166c is shaped to permit the IOL and the plunger to exit the holder and enter the lumen of the distal portion 130 of injector 100. In some embodiments, it is advantageous if holder 160 is constructed such that hole 166c is located close to open end 164 so that the lens enters the distal portion of the lumen immediately upon exiting the container. An advantage of such embodiments is that there is a reduced risk that the IOL is misaligned during actuation of the plunger, and the IOL is delivered to an eye in an improper orientation. Other embodiments of a container and corresponding proximal and distal body portions are given in an application titled INTRAOCULAR LENS INJECTOR APPARATUS AND METHODS OF USE, by inventor J. Pynson filed on even date herewith, Attorney Docket No. P03425, the substance of said application is hereby incorporated by reference herein.

According to some aspects of the invention, IOL injector 100 has a proximal cutting surface 135 disposed proximate an end of proximal body portion 120 and extending distally of lumen 121 (e.g., into gap 145). In some embodiments, it is advantageous if the proximal cutting surface is coupled to the proximal body portion. In some embodiments, the proximal cutting surface is attached to or integrally formed with the proximal body portion. IOL injector 100 also has a distal cutting surface 125 disposed proximate an end of the distal portion and extending proximally of lumen 131 (e.g., into gap 145). In some embodiments, it is advantageous if the distal cutting surface is coupled to the distal body portion. In some embodiments, the distal cutting surface is attached to or integrally formed with the distal body portion.

It is to be appreciated that the term “cutting surface” includes a surface of any suitable shape for cutting an end cover 163, 165. Preferably, the cutting surface forms a sharp edge. For example, a cutting surface may be shaped as a blade as illustrated in FIG. 1 or may be shaped as a needle (i.e., having a thin, pointed shape).

As described in greater detail below, container 150 is configured such that proximal cutting surface 135 and distal cutting surface 125 cut end cover 163 and end cover 165, respectively, as container 150 is moved into place between the proximal body portion and the distal body portion. In some embodiments, it is advantageous if proximal open end 162 and distal open end 164 of container 150 are angled relative to one another (i.e., they are not parallel to one another) to facilitate contact of the end cover 163 and end cover 165 with respective cutting surfaces 125, 135. In some embodiment, both open ends, 162 and 164 are angled relative to the longitudinal axis of the container lumen. However, open ends 162 and 164 need not be angled relative to one another to achieve advantages of some aspects of the present invention. In embodiments where the open ends are angled, it may be advantageous to angle the end of distal body portion 130 that is proximate gap 145 (i.e., the proximal end of distal body portion is angled so as to be other than perpendicular to axis A-A) and/or to angle the end of proximal body portion 120 that is proximate gap 145 (i.e., the distal end of proximal body portion is angled so as to be other than perpendicular to axis A-A) to facilitate contact of the end cover 163 and end cover 165 with respective cutting surfaces 125, 135.

It is to be appreciated that although lumens 121, 131 and lumen 154 are illustrated as rectangular and square respectively, the shapes of the lumens may be any other suitable shapes such as round or polygonal. Furthermore, lumens 121, 131 and 154 may have shapes that are different than one another. In some embodiments, the lumen of the container at open end 162 and/or at open end 164 is larger than the lumen of the distal body portion and the lumen of the proximal body portion. However, such a construction is not necessary and the lumen of the container at open end 162 and/or at open end 164 can be similarly sized or smaller than a corresponding one of the distal body portion and the proximal body portion.

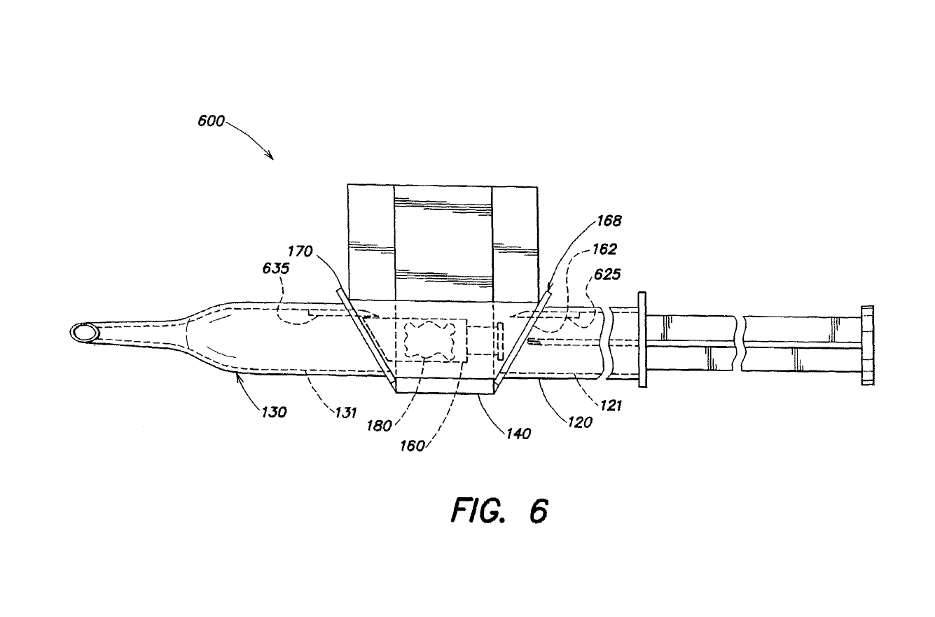

Although cutting surfaces 125 and 135 are illustrated in FIG. 1 as being disposed outside of a profile of lumen 121 and of lumen 131, respectively, in some embodiments it is advantageous that one or both of cutting surfaces 125 and 135 be disposed inside of a profile of a respective one of lumens 121 and 131. For example, FIG. 6 is a top view of an alternative embodiment of an injector 600 according to aspects of the invention in which cutting surfaces 625 and 635 are located inside of a profile of proximal lumen 121 and inside of a profile of distal lumen 131, respectively. That is, in FIG. 6, if the distal lumen were extended further in a proximal direction, it would enclose cutting surface 635; and similarly, if the proximal lumen were extended further in a distal direction, it would enclose cutting surface 625

It is to be appreciated that an advantage of locating a cutting surface inside of a profile of a lumen is that more intimate contact can be achieved between the container and a corresponding body portion (e.g., a more intimate contact can be achieved by a frame 168 and a frame 170, and an end of a respective one of proximal body portion 120 and distal body portion 130). Other than placement of the cutting surfaces, injector 600 is the same as injector 100 discussed above.

Referring again to FIG. 1, the injector body (including the cutting surfaces), the container and/or the holder may be constructed of any suitable material. For example, a material that is sterilizable and has desirable molding characteristics, such as polypropylene, may be used. In some embodiments, it is advantageous if the injector body, the container and/or the holder are constructed of a transparent material. The end covers may, for example, be made of a suitable foil material.

The container, holder and injector body may be constructed to operate with a single-element IOL or a multi-element IOL. The single-element or multi-element may have any suitable construction.

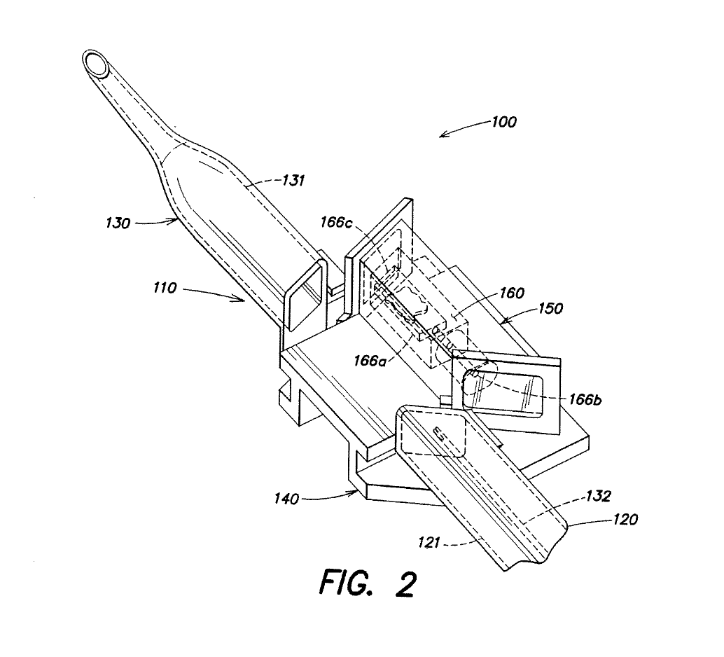

FIG. 2 is a perspective view of the embodiment of the IOL injector of FIG. 1, in which injector body 110 and container 150 are interacting to approach a complete IOL injector lumen. For example, the interaction may comprise sliding the container along connector 140 to reach a position in which the lumen of injector 100 is complete. In embodiments in which a guide is provided, the interaction may comprise sliding the container along the guide. For example, in some embodiments, a bottom surface of each of covers 163 and 165 or a bottom portion of container 150 may be sized to contact and slide along a guide 146, thereby permitting alignment of lumens 121, 131 and formation of a complete IOL injector lumen. It is to be appreciated that such sliding, while advantageous in some embodiments, is not necessary according to aspects of the invention.

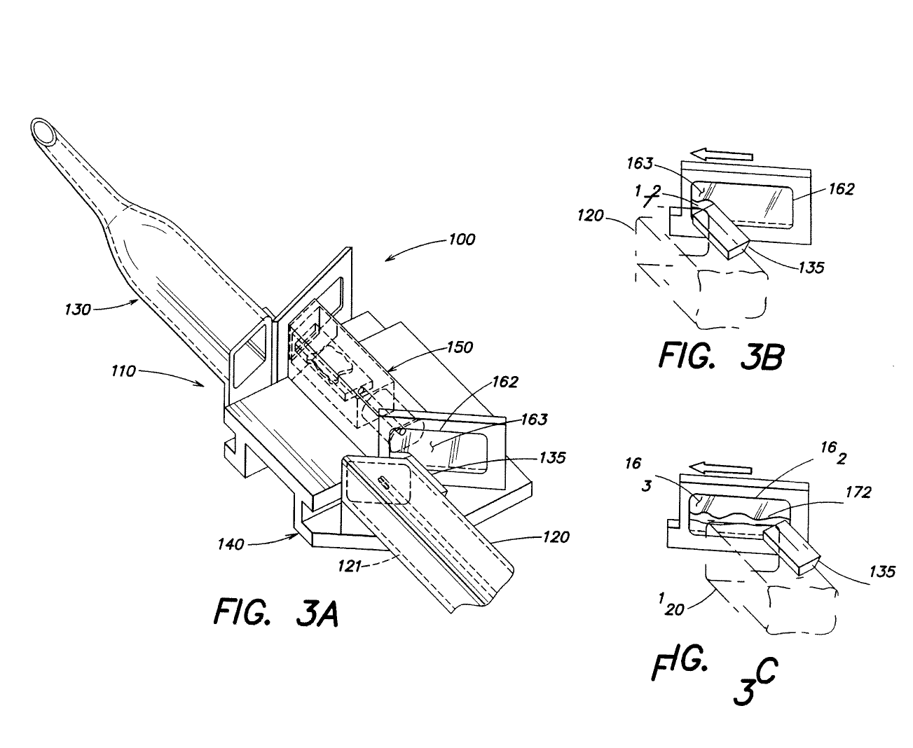

FIG. 3A is a perspective view of the embodiment of the IOL injector of FIG. 1 in which the injector body and the container are interacting to approach a complete IOL injector lumen, and the cutting surfaces of the injector body are cutting the end covers of the container. That is, during a process of moving the container into a position proximate the injector body, the first end cover and the second end cover are cut by the first cutting surface and the second cutting surface, respectively.

It is to be appreciated that in some embodiments (not shown) proximal cutting surface 135 is omitted. In such embodiments, only the distal end cover 165 is cut by a cutting surface (i.e., cutting surface 125) as the container is moved into a position proximal the injector body. In such embodiments, for example, proximal end cover 163 may be breached by plunger tip 134 as the plunger is actuated.

FIGS. 3B and 3C are expanded views of the interaction between the proximal cutting surface of the injector body, and the proximal end cover of the container to form a tear 172. In FIG. 3B, the cutting interaction between the cutting surface and the end cover is beginning to form a tear 172; and in FIG. 3C the cutting interaction is substantially complete, as a result of the relative motion of the container and the injector body. In FIGS. 3B and 3C, proximal body portion 120 is partially removed to avoid obscuring the view of the container.

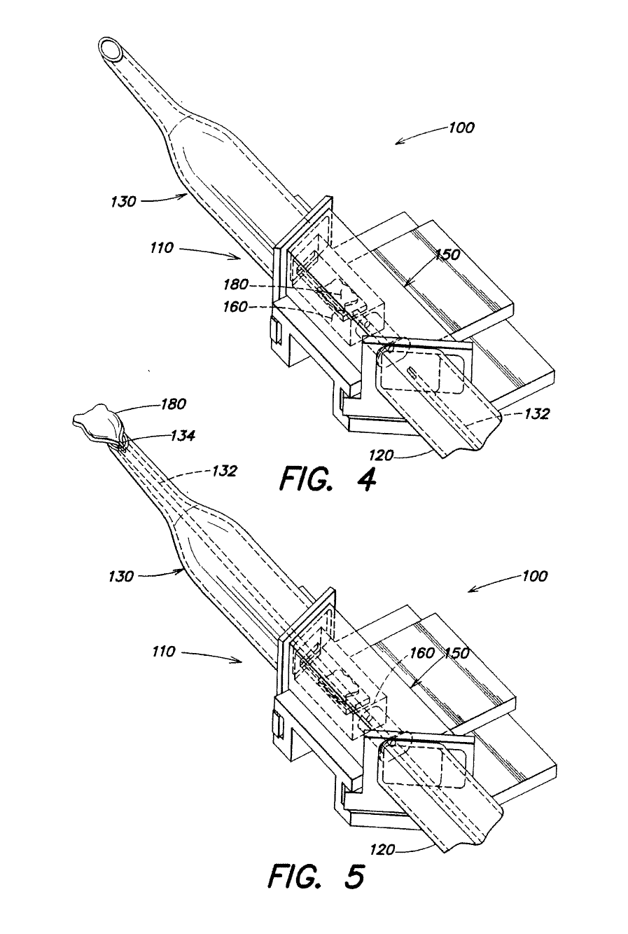

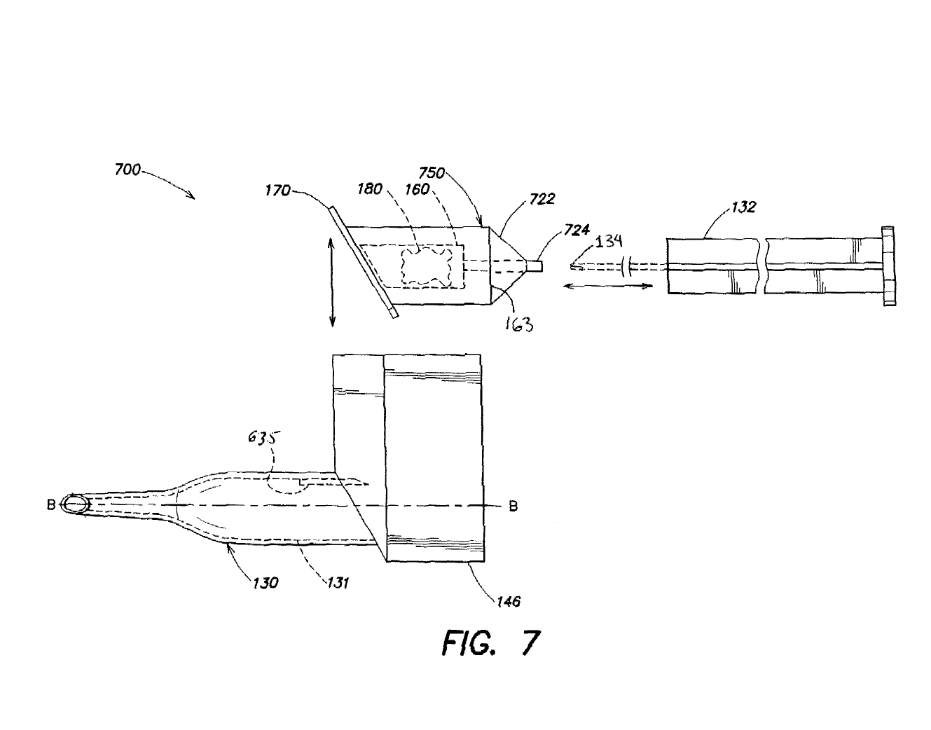

FIG. 4 is a perspective view of the IOL injector of FIG. 1 in which a complete injector lumen is formed by the injector body and the container. The term “complete injector lumen” means that the lumen of the proximal body portion, the lumen of the distal body portion, and the container lumen are aligned such that the plunger can extend through each of the lumens. To achieve a complete injector lumen, the container was moved into a position proximate injector body 110 which is configured to define an injector body lumen, such that the container lumen and the injector body lumen at least partially overlap. As a result of the overlap, a plunger can extend through the injector body lumen and the container lumen. It is to be appreciated that the injector body lumen can include the distal body lumen 131 and the proximal body lumen 121 (see FIG. 1) although, as described below (see FIG. 7), the injector body lumen may omit the proximal body portion and corresponding lumen.

In some embodiments, when the container is in a final position (i.e., a position in which the plunger is to be actuated), the center of the container lumen is aligned with either one or both of the center of the lumen of distal body portion and the center of the lumen of the proximal body portion. In other embodiments, when the container is in a final position the container lumen is not aligned with either one or both of the center of the lumen of distal body portion and the center of the lumen of the proximal body portion.

In some embodiments, the proximal and/or distal body portions and the container may be configured such that coupling the container to the injector body comprises snapping the container into a final position. Alternatively, connector 140 or guide 146 may be configured such that container snaps into a final position thereby coupling the container to the injector body. In such embodiments, a snap feature may provide robustness to the injector prior to and during actuation of the plunger.

FIG. 5 is a perspective view of the IOL injector 100 of FIG. 1 in which the IOL 180 is illustrated as being ejected from the completed injector lumen by actuating the plunger 132.

FIG. 7 is a top view of another example of an embodiment of an IOL injector 700 according to aspects of the present invention in which an injector body 130 and a container 750 are separated from one another. The injector body has an axis B-B extending through distal body portion 130. Plunger 132 is moved through lumen 131 along axis B-B to insert an IOL into an eye.

Because container 750 is configured with plunger holding apparatus 724 to maintain plunger 132 during actuation to push an IOL 180 into an eye, a proximal body portion is not necessary. The plunger holding apparatus can be coupled to container 750 using any suitable structure 722. Distal cutting surface 125 is disposed proximate a proximal end of the distal portion and extends in a proximal direction of said first lumen (into a space into which container 750 is to be received). A guide 146 may be provided to couple to container 750. Container 750 can be configured similarly to container 150 except for the addition of plunger holding apparatus 724. The location of distal cutting surface 125 is selected to facilitate cutting of distal end cover 165 as container 750 is coupled to distal body portion 130. Proximal end cover 163 can be breached by tip 134 of plunger 132 or by another suitable technique.

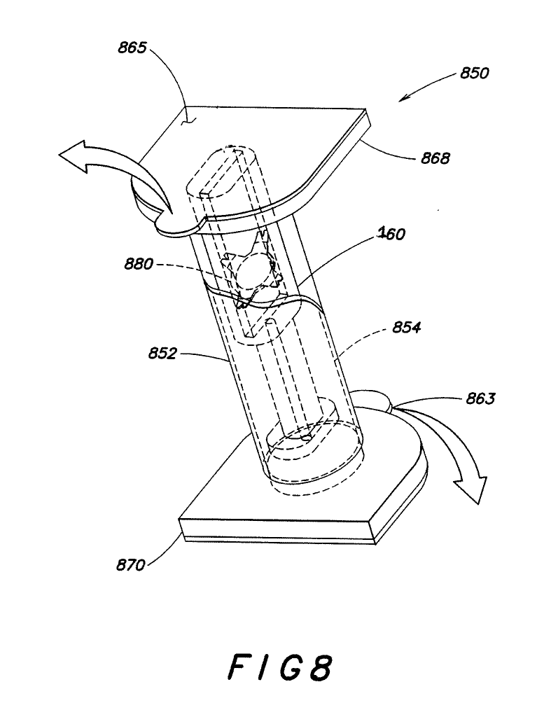

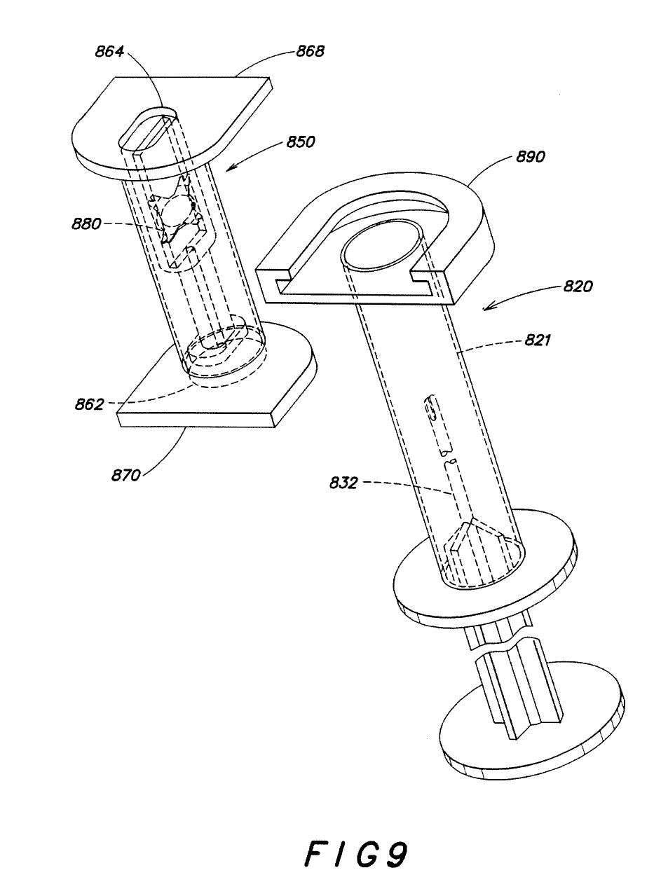

FIGS. 8 and 9 are perspective views of another embodiment of an IOL container 850. FIG. 9 also includes a proximal body portion 820. Container 850 is particularly suitable for use with hydrophilic IOLs because of the ability of the container to maintain fluid. As illustrated in FIG. 11, the container comprises a first connector 868 and a second connector 870 capable of engaging the container to a distal body portion 830 and proximal body portion 820, respectively.

Referring again to FIGS. 8 and 9, container 850 comprises a wall 852 defining a container lumen 854, a proximal open end 862, and a distal open end 864. A proximal end cover 863 and a distal end cover 865 are configured and arranged to seal said first open end and said second open end, respectively.

A hydrophilic IOL 880 is disposed inside container 850. It is to be appreciated that, by sealing container 850, IOL 880 can be maintained sterile and/or hydrated. To maintain hydration, a fluid is sealed inside the container along with the IOL. Typically, the fluid is provided in sufficient quantity such that the IOL is immersed in the fluid regardless of the orientation of the container.

IOL 880 may be maintained in a location within the container by any suitable apparatus such that the plunger tip can urge the IOL into an eye upon actuation, after formation of an injector. For example, IOL 880 may be disposed in a holder 160 as was discussed above with reference to FIG. 1. (Wall 852 is illustrated as partially cut away so that holder 160 is visible.)

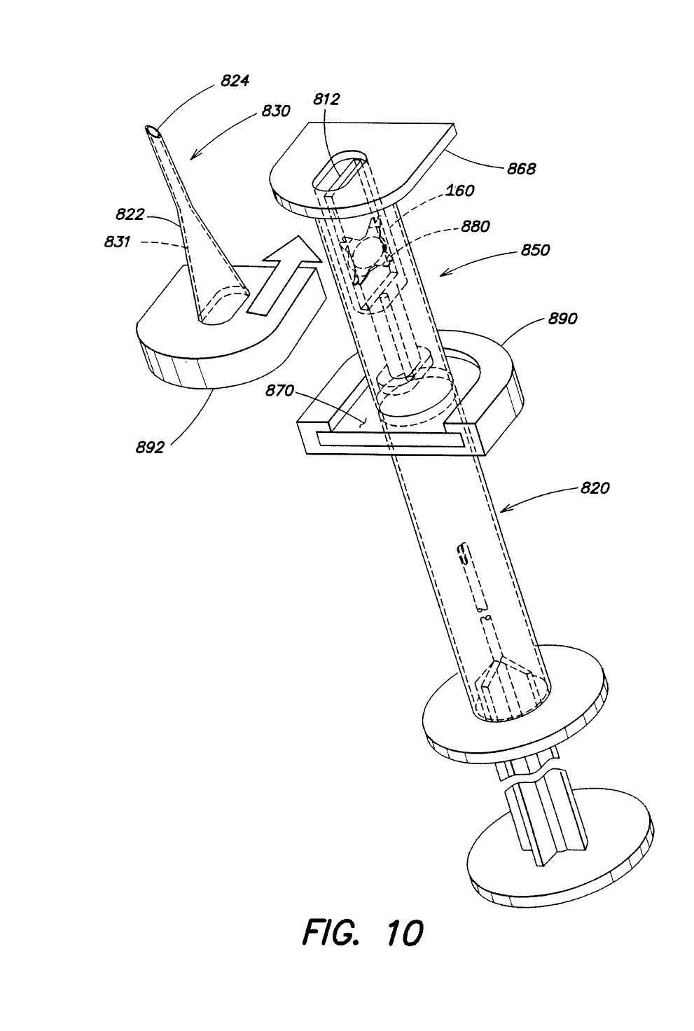

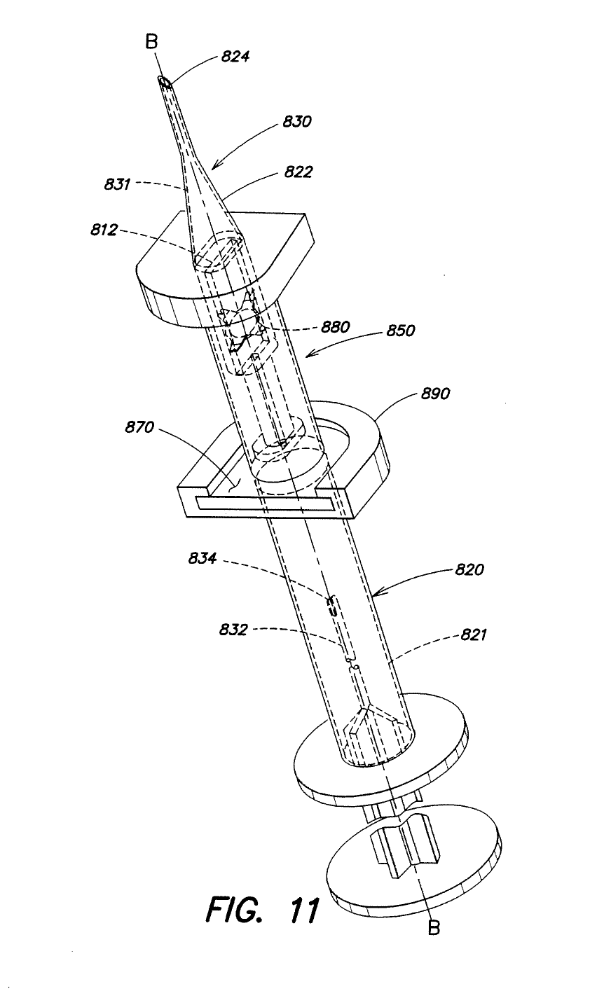

FIG. 10 is a perspective view of the IOL container of FIG. 8 and a proximal body portion 820 and a distal body portion 830 of an IOL injector. Proximal body portion 820 comprises a first lumen 821 disposed therethrough along a first longitudinal axis; and distal body portion 830 comprises a second lumen 831 disposed therethrough along a second longitudinal axis.

Proximal body portion 820 and container 850 (e.g., a vial) are capable of being engaged to one another using connectors 870 and 890; and distal portion 830 and container 850 (e.g., a vial) are capable of being engaged to one another using connectors 868 and 892. In FIG. 10, the proximal body portion and the container are engaged to form a partial injector.

Pair of connectors 870 and 890, may be configured such that the proximal body portion and the container engage one another using any suitable connectors. For example the connectors may achieve a bayonette connection, a threaded connection or a tongue-in-groove connection (shown in FIGS. 8-11). Similarly, pair of connectors 868 and 892, may be configured such that the distal body portion and the container engage one another using any suitable connectors. For example the connectors may achieve a bayonette connection, a threaded connection or a tongue-in-groove connection. The connectors may be attached to a respective one of the proximal body portion, the distal body portion and the container or may be integrally formed therewith. After engagement of connectors 870 and 890, and connectors 868 and 892, the proximal body portion, the distal body portion and the container are rigidly connected together to form an injector.

In some embodiments (e.g., where the connectors form a bayonette-type connection), container 850 and each of proximal body portion 820 and distal body portion 830 are configured such that the container slidably engages with each of the proximal body portion and the distal body portion in a direction transverse to a longitudinal axis extending through the lumens of the body portions. Also, the container, and each of the proximal body portion and the distal body portion are configured to provide a fixed end stop of the slidable engagements. The fixed stop features align the lumens of the body portions with the container lumen. The phrase “transverse to the optical axis” means other than parallel to the optical axis. In some embodiments it is advantageous if transverse engagement is 45-90 degrees to the optical axis; in other embodiments it is advantageous if transverse engagement is 60-90 degrees to the optical axis; in other embodiments it is advantageous if transverse engagement is 80-90 degrees to the optical axis; and in some embodiments it is advantageous if transverse engagement is at about 90 degrees to the optical axis or is 90 degrees to the optical axis.

In some instances, it is advantageous if both of the covers 863 and 865 are removed prior to connecting of the container with either of the proximal body portion or the distal body portion. However, according to some assembly techniques it is advantageous if the covers are not removed in such a manner. One example of such a technique is performed as follows. With container 850 in an orientation as illustrated in FIG. 8 (i.e., proximal cover 863 facing up) cover 863 is removed. Proximal body portion 820 is then attached to the container to form a partial injector. Subsequently, the partial injector can be inverted (i.e., oriented such that cover 865 faces up), and cover 865 removed. Distal portion 830 is then attached. Proximal body portion 820 may be provided with any suitable fixturing (not shown) to maintain all or a substantial portion of the fluid in the injector when the injector is inverted.

FIG. 11 is a perspective view of IOL container 850, a proximal body portion and a distal body portion assembled to form a completed injector lumen. The distal body portion includes a cartridge 822 suitably shaped to fold and/or compress an IOL as the IOL passes through the cartridge upon actuation of a plunger 832. The IOL passes through the cartridge to an end 824 where the IOL is delivered into an eye. Although the illustrated embodiment of the IOL injector includes a cartridge to fold and/or compress the IOL, an injector according to aspects of the present invention may be provided with no such apparatus. For example, a compressor drawer may be included to fold and/or compress the IOL.

Axis B-B extends through the first lumen and the second lumen. Upon assembly of the inserter, the first and second lumens are aligned such that plunger 832 can extend through the first and second lumens. In some embodiments, the lumens are configured and arranged such that axis B-B extends substantially through the centers of the container, the proximal portion, and the second portion. However, such an alignment is not necessary, and the centers may be offset from one another.

As illustrated in FIGS. 10 and 11, the proximal body portion has a connector 890 and distal body portion has a connector 892 that are capable of interacting with connectors 870 and 868 of the container, respectively. In particular, in the illustrated embodiment, the distal end of the container (i.e., the connector 868) and the distal body portion (i.e., the connector 892) are configured to slidably engage one another in a direction transverse to longitudinal axis B-B and configured to provide a fixed end stop to define an alignment of the first lumen and the container lumen. Also, in the illustrated embodiment, the proximal end of the container (i.e., the connector 870) and the proximal body portion (i.e., the connector 890) are configured to slidably engage one another in a direction transverse to longitudinal axis B-B and configured to provide a fixed end stop to define an alignment of the first lumen and the container lumen. In some embodiments, it is advantageous if the connectors 890, 892, 868 and 870 provide a seal to maintain the fluid in the injector. Such a seal may be achieved, for example, with a tongue-in-groove connector arrangement (as shown in FIGS. 8-11), a threaded connector arrangement, a bayonet connector arrangement or a connector arrangement including one or more snaps.

During actuation of the plunger (not shown), a tip 834 of the plunger pushes the IOL through the distal portion of the lumen and into an eye through end 824. It is to be appreciated that proximal body portion 820 may have any suitable configuration such that a plunger is maintained prior to actuation, and such that the plunger can be actuated to push an IOL into an eye.

In some embodiments, the components of a kit may be packaged in a single sterile container. In other embodiments, a plurality of sterile containers may be used, each maintaining one or more components. In embodiments, in which a plurality of sterile containers is used, the sterile containers may be further packaged in a non-sterile container such as a cardboard container.

An advantage of an injector according to the present aspects of the invention is that the construction provides versatility. In some embodiments, to take advantage of the versatility, the parts are provided in a kit comprising a single proximal portion and a single distal portion along with plurality of containers each including a lens. For example, the plurality of lenses may be made of different materials or have different optical powers or have other distinctions.

Another advantage of an injector according to the present aspects of the invention is that the construction is economical. For example, due to the differing lifespans of the proximal body portion (which may, for example, comprise a lubricious material), the container (which contains a lens and a liquid), and the proximal portion which has neither a liquid or lubricious material, it may be advantageous to produce the components 820, 830 and 850 (with an IOL disposed therein) at differing times and in differing quantities.

Having thus described the inventive concepts and a number of examples of an embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An IOL injector comprising:
A) an injector body, comprising
(i) a first body portion having a first lumen disposed therethrough, a proximal end, a distal end configured to deposit an IOL into an eye, and
(ii) a first cutting surface coupled to the first body portion, the first cutting surface being disposed proximate the proximal end of the first body portion and extending proximally of said first lumen; and
B) a plunger coupled to the injector body, the plunger having a longitudinal axis.

2. The injector of claim 1, wherein the first body portion is suitably shaped to fold the IOL as the IOL passes therethrough.

3. The injector of claim 1, further comprising a guide coupled to the first body portion configured to facilitate coupling of a container to the injector.

4. The injector of claim 1, wherein the injector body includes an attachment device such that a container can be connected to the injector body.

5. The injector of claim 4, wherein the first cutting surface is attached to the first body portion.

6. The injector of claim 5, wherein the first cutting surface is integrally formed with the first body portion.

7. The injector of claim 1, wherein the proximal end of the first body portion is angled so as to be other than perpendicular to the axis.

8. The injector of claim 1, wherein the first cutting surface is disposed inside of a profile of the first lumen.

9. The injector of claim 1, further comprising a second body portion having a second lumen disposed therethrough, the second body portion and the first body portion being separated along the axis by a gap, the second body portion being disposed in a location that is more proximate than the first body portion, and the first and second lumens aligned such that the plunger can extend through the first and second lumens.

10. An IOL injector comprising:
A) a plunger having a longitudinal axis; and
B) an injector body, comprising
(i) a proximal body portion having a first lumen disposed therethrough and a distal body portion having a second lumen disposed therethrough, the proximal body portion and the distal portion being separated along the axis by a gap, and the first and second lumens aligned such that the plunger can extend through the first and second lumens, and
(ii) a distal cutting surface disposed proximate an end of the distal portion and extending into said gap.

11. The IOL injector of claim 10, further comprising a proximal cutting surface disposed proximate an end of the proximal body portion and extending into the gap.

12. The IOL injector of claim 10, further comprising a connector configured and arranged to couple the proximal body portion and the distal body portion together.

13. The IOL injector of claim 10, further comprising a guide configured to facilitate movement of a container into a position between the proximal body portion and the distal body portion.

14. The IOL injector of claim 13, wherein the guide is attached to a connector, the connector being configured and arranged to couple the proximal body portion and the distal body portion together.

15. The IOL injector of claim 14, wherein the guide is integrated with the connector.

16. The IOL injector of claim 13, wherein the guide is disposed on at least one of the proximal and distal body portions.

17. The IOL injector of claim 11, wherein at least one of the cutting surfaces is disposed inside a profile of a respective one of the first and second lumens.

18. The IOL injector of claim 10, wherein the proximal body portion is rotationally complete.

19. The IOL injector of claim 10, wherein at least one of the proximal end of the distal body portion and the distal end of the proximal body portion is angled so as to be other than perpendicular to the axis.

20. A container for use with an IOL injector body, comprising:
a wall defining a container lumen, a first open end, and a second open end;
a first end cover and a second end cover configured and arranged to seal said first open end and said second open end, respectively; and
an IOL disposed inside said container.

21. The container of claim 20, wherein the IOL is disposed in a holder, the holder being disposed in the container.

22. The container of claim 20, wherein the first open end and the second open end are angled relative to one another.

23. The container of claim 20, further comprising a plunger holding apparatus.

24. A method of loading an IOL injector with an IOL, comprising:
providing a sealed container containing an IOL, the container comprising a first end cover to seal a first end of the container and a second end cover to seal a second end of the container, and a container lumen;
moving the container into a position proximate an injector body defining a first injector body lumen such that the container lumen and the first injector body lumen at least partially overlap such that a plunger could extend through the first injector body lumen and the container lumen; and
during the step of moving the container into a position proximate the injector body, cutting at least one of the first end cover and the second end cover with a first cutting surface coupled to the injector body.

25. The method of claim 24, wherein the step of moving the container further comprises sliding the container along a guide.

26. The method of claim 24, further comprising breaching the other of the first end cover and the second end cover with a plunger tip.

27. The method of claim 24, further comprising snapping the container into a final position relative to the injector body.

28. The method of claim 24, further comprising moving the container into a position proximate a second injector body lumen such that the container lumen and the second injector body lumen at least partially overlap such that a plunger could extend through the first and second injector body lumens and the container lumen.

29. The method of claim 28, further comprising a step of, during the step of moving the container into a position proximate the injector body, cutting the other of the first end cover and the second end cover with a second cutting surface coupled to the injector body.
